(19) United States
(12) Patent Application Publication
(10) Pub. No.: US 2021/0121833 A1
(43) Pub. Date: Apr. 29, 2021

(54) Producing Homogeneous Metastable Solutions from Heterogeneous Non-condensed Solutes And Liquid Solvents

(71) Applicant: C. Edward Eckert, New Kensington, PA (US)

(72) Inventor: C. Edward Eckert, New Kensington, PA (US)

(21) Appl. No.: 16/665,057

(22) Filed: Oct. 28, 2019

(51) Int. Cl. B01F 3/04; B01F 7/00; B01F 7/06; B01F 15/02; B01F 15/00; A61K 33/00; A61K 9/08

(52) U.S. Cl. CPC B01F 3/04588; B01F 7/0025; B01F 7/00341; B01F 7/06; B01F 15/0283; B01F 2215/0431; B01F 15/00344; A61K 33/00; A61K 9/08; B01F 2003/04631; B01F 2215/0034; B01F 15/00188

(57) ABSTRACT

Disclosed is a method and a device for producing a homogenous metastable supersaturated solution. The homogenous metastable supersaturated solution is comprised of a liquid phase solvent and one or more solutes wherein these individual solutes are gases under conditions of standard temperature and pressure (STP) in a normal atmospheric environment. When the device is used for creating oxygen saturated metastable solutions, the oxygen levels reached to at least 3 times the maximum level that can be detected by commercial dissolved oxygen probes. The device can be used to treat any medical condition that can benefit from the use of high oxygenated or any other highly saturated gas solutions. The device can be used for any non-medical need where a highly saturated gas in solution is needed.

### BACKGROUND

In the medical and veterinary community, the effect of oxygen on living tissue is generally characterized by one of three regimes: metabolic enhancement (growth acceleration), metabolic inhibition (growth arrest), and toxicity. Oxygen has been used to accelerate the healing and regeneration rate of damaged tissue for wounds such as cuts, lacerations, sores and burns on the body. When these wounds begin to heal, fibroblastic cells divide and spread throughout the wound area. These fibroblastic cells produce collagen, an important protein that facilitates healing. Supplying sufficient quantities of oxygen to the wound area significantly enhances fibroblast proliferation.

In addition to treating wounds, oxygen is used in topical applications for cleaning and revitalizing skin. In facial cleansing, dissolved oxygen assists in exfoliating dead particles from the skin surface. Dissolved oxygen has also been used to remove toxins, particulates and other occlusions in skin pores. Further, oxygen may be able to oxidize oils in the skin pores, thus allowing the pores to be backfilled with water and become receptive to infiltration by beneficial lotions and other skin care products. Without the oxidative effects, pores in the skin would remain filled with oil that would require displacement from the pore before lotions could occupy pore volume. Furthermore, oxygen has been used to revitalize skin cells by aiding in the production of collagen. It is even possible that dissolved oxygen can stimulate hair follicles and consequentially hair growth.

Improvement in skin topography (roughness) has been also observed following exposure of the skin to oxygen dissolved in water. Stereoscopic examination of the skin indicates that the peaks that exist in the epidermal layer of the skin become smooth; presumably as a result of selectively higher oxidation rates associated with the higher surface area ridges of the skin. Another use for a highly oxygenated solution is to oxygenate hemoglobin via the peripheral capillaries of humans and animals. Increasing blood oxygen content has many benefits including, but not limited to headache relief, improved circulation, and relief of muscle stiffness.

Oxygen is of great interest in oncological applications. Research indicates that oxygen may beneficially influence DNA methylation patterns. Further, mitochondrial activity in malignant cells appear to strongly favor anaerobic glycolysis in lieu of the more energetic aerobic process of oxidative phosphorylation or citric acid cycle; i.e.: the Warburg Effect. Oxygen availability at the cellular level therefore promotes normal cell proliferation while suppressing malignant cell growth.

Research indicates that oxygen promotes the development of new blood vessels from existing ones (angiogenesis) and the de novo development of new vessels (vasculogenesis). These mechanisms can significantly enhance, for example, wound healing, and peripheral blood flow in compromised individuals such as diabetic patients.

A specialized ophthalmological application using oxygen may be in treating retinal ischemia. Here, water or other fluid to maintain osmotic balance is enriched with dissolved molecular oxygen to a specific concentration. This oxygenated solution is brought into contact with the eye, with the intention of diffusing molecular oxygen through the cornea, anterior chamber, iris, and into the posterior segment through vitreous humor. From here, oxygen may transport into the choroid.

Whole body hyperbaric oxygen therapy (HBOT), is applied in 30 to 150 minutes sessions within a hyperbaric chamber and has been shown to improve wound healing for a variety of patients, including those that are diabetic without overt ischemia. HBOT chambers deliver pure gaseous oxygen at up to 2.8 atmospheres (absolute) pressure resulting in oxygen concentrations higher than ambient conditions by a factor of approximately 13.3. Equipment and patient considerations limit HBOT operating pressures to this value. Since the kinetics of oxygen dissolution at the skin surface are extremely sluggish, oxygen delivery to wound tissues is accomplished primarily by increasing the blood plasma oxygen tension through respiratory uptake. Gaseous oxygen absorption through the wound is also minimal. Various attempts to locally envelop a wound site with pure topical oxygen gas using flexible and rigid containment systems have been developed, but not proven effective.

Conventional HBOT has disadvantages. First, profusion is required to deliver oxygen at the cellular level in tissue, particularly in the case of the extremities. Oxygen delivery requires adequate blood flow. Ischemic conditions therefore typically result in hypoxic conditions. In situations where peripheral blood flow is compromised, such as diabetes, HBOT depends on circulation for oxygen delivery.

Second, a theoretical HBOT oxygen delivery limitation exists with respiratory oxygen uptake as HBOT is currently practiced. Since oxygen solubility and PO₂ are related through a linear function, an alveolar PO₂ of 13.3 times that of ambient air (2.8 atmospheres with 100% oxygen compared to 21% oxygen in ambient air) will only increase blood plasma oxygen tension from a base of 0.37 cm³ O₂/dL (˜6 mg/l) to 4.95 cm³ O₂/dL (˜80 mg/l). Moreover, hemoglobin is typically fully saturated under ambient conditions, representing about 20 cm³ O₂/dL unless COPD or anemia is present. The total theoretical respiratory oxygen uptake under conventional hyperbaric chamber uptake conditions is therefore 24.95 cm³ O₂/dL blood compared to a base value of 20.37 cm³ O₂/dL, representing an increase of only 24.3%.

Third, the capital cost for a conventional HBOT chamber is reported to approach $1 MM. The rather large equipment cannot be easily transported and is certainly not amenable for home-based patient care. All patients, including impaired, compromised, and non-ambulatory individuals must be transported to a HBOT center, relegating HBOT to a remedial, rather than prophylactic, role. Additionally, inherent equipment and physiological dangers exist with conventional HBOT.

The amount of oxygen initially dissolved into solution is largely dependent on the method used to dissolve the oxygen gas into solution. Generally, these methods consist of two steps: creating a solute gas/solvent liquid interfacial area, and, exposing the gas/liquid mixture to elevated pressure. The former step affects the kinetics or rate at which the solution process occurs, while the latter determines the maximum theoretical dissolved. Small bubbles create interfacial area and promote more favorable kinetics. The second step is a pressure-concentration relationship, such as Henry's Law for dilute solutions and Sievert's Law for diatomic gases at higher concentrations. These steps may be combined, although the source of oxygen must operate at a higher final pressure rather than allowing a pump, for example, to pressurize both the liquid and gas components after the gas has been introduced.

One common method for oxygenating water is the coarse bubble aeration process, which is a subset of aeration methods known categorically as air diffusion. Coarse bubble aeration is frequently used in bulk situations such as waste water treatment or aquaculture where high throughput and comparatively low dissolved oxygen (DO) values are required. Pressurized air or oxygen gas is introduced through a submerged pipe having small holes or orifices into a container of water. Gas pressure is sufficient to overcome the hydrostatic head pressure, and also sustains pressure losses during passage through the small gas orifices. As a result, bubble aeration occurs at relatively low pressures; this pressure being predominantly a function of tube immersion depth and density of the liquid, which in this case is water. Coarse bubble aeration is unsuitable for medical applications.

Various derivative methodologies related to coarse bubble aeration use diffusers and jets to facilitate higher oxygenation efficiencies; that is, greater in-solution oxygen recovery. The use of these devices is motivated by the need to create gas/liquid interfacial area for oxygen dissolution. A so-called “phase contactor” is a device that accomplishes this task. As will be discussed in detail later, the development of such surface area is an energy intensive process. The exclusively pneumatic processes based on diffusers and jet rely on a pressure—surface energy conversion and the isothermal expansion energy of the gas. Many of these methodologies based on pneumatic energy sources are very limited in their ability to create gas/liquid interfacial area based on available energy inherent with such previous phase contactors. Typically, they produce a plume of small (or commonly referred to as “microbubbles”) that is suspended within a water outflow. This plume can indeed provide a measurable DO of up to 40 mg/l since this value represents the DO equivalent concentration of pure oxygen at atmospheric pressure.

Oxygen dissolution in bubbling aeration is also limited by ambient pressure conditions above the solution; hyper-saturated solutions with DO values above approximately 40 mg/l of oxygen in true solution form are not possible using conventional systems. However, realistically the highest concentration of oxygen in a solution for conventional systems is 35 mg/l. Further, if the solution being oxygenated is subsequently exposed to atmospheric conditions, the dissolved oxygen concentration will be limited to the solubility limit of oxygen (at its partial pressure in air of 0.21 atm) under such conditions; typically, less than 10 mg/l. The desirability of bubbling aeration is further hampered by equipment and energy requirements. Large blower units are used to force the gas bubbles into the carrying liquid. These blowers generate high-energy costs and often require special soundproof installations or other engineering costs. Bubble aeration is therefore an impractical process for producing oxygenated solutions or solution/suspensions for health-related applications.

What is needed is a device and method where oxygen hypersaturation of liquid (a hypersaturated aqueous solution) can be reliably achieved clinically. Such a device should be easily capable of consistently and safely achieving therapeutic DO values on the order of 80-100 mg/l, or approximately equivalent to a predicate HBOT chamber. Since DO values in HBOT are limited by equipment and patient pressure tolerance, the ideal device should be capable of exceeding traditional HBOT limitations of 80-100 mg/l equivalent DO. Greater clinical benefit is expected from higher DO values. Again, it must be emphasized that a true solution of dissolved oxygen is needed and not a small or microbubble suspension that is limited to about 35 mg/l DO produced by conventional systems. Treatment with this oxygenated hypersaturated liquid exceeds the effectiveness HBOT with DO values essentially unencumbered by system and patient limitations. And unlike HBOT, a hypersaturated aqueous solution (HSAS) system does not require extraordinary safety considerations since the dominant phase is water. The facilities or maintenance needed for effective oxygen therapy are not complex nor is there the high cost that is characteristic of existing gas based HBOT.

While certain aspects of conventional technologies have been discussed to facilitate disclosure of the invention, Applicants in no way disclaim these technical aspects, and it is contemplated that the claimed invention may encompass one or more of the conventional technical aspects discussed herein, especially in combination with the innovative aspects described herein.

The present invention may address one or more of the problems and deficiencies of the art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### SUMMARY OF THE INVENTION

Disclosed is a method and a device for producing a homogenous metastable supersaturated solution. The homogenous metastable supersaturated solution is comprised of a liquid phase solvent and one or more solutes wherein these individual solutes are gases under conditions of standard temperature and pressure (STP) in a normal atmospheric environment. The solubilized concentration of at least one solute gas is a minimum of a 1.1 multiple above the concentration of the saturation conditions of this gas in the particular solute liquid at STP conditions. The claimed invention is capable of producing a solution wherein least one solute gas is more typically greater than 3 times the concentration at STP saturation conditions, i.e. greater than a hyperbaric chamber.

Methodologically, a liquid at a desired pressure flows into a phase contactor. The liquid may be densified using a densification pump. Simultaneously or sequentially, one or more solute gases from a gas source are introduced into the phase contactor at a desired rate. The gas flow rate is preferably controlled by a mass flow controller. The gas is at a pressure above the liquid. Within the phase contactor a highly densified, interfacial gas/liquid dispersion is created. This dispersion flows into a pressurization pump to create a solution with the desired gas solute concentration i.e. the nascent solution. Care is taken to avoid coalescence of the dispersed phase while the dispersion flows into the pressurization pump. The pressure of the nascent solution is reduced from the pressure used to form it, to the pressure intended for its use without substantially reducing the solute gas concentration by creating zero microbubbles. One means of reducing the pressure is to have the nascent solution flow through a stepless depressurization tube transforming the nascent solution into a metastable solution.

When the device is used for creating oxygen saturated metastable solutions, the oxygen levels achieved to at least 3 times the maximum level that can be detected by commercial dissolved oxygen probes. The oxygen saturated metastable solution described herein has been used in baths healing the wounds and lesions in preliminary animal studies. Additional studies of the metastable solutions created by the certain embodiments of the device also been used in cellular level oncological experiments, ischemic conditions in rats, and in-vitro tissue preservation and grafting using mice. Limb/organ preservation, hair follicle stimulation, angiogenesis and vasculogenesis are also contemplated by this disclosure. The device being used to treat any medical condition that can benefit from the use of high oxygenated or any other highly saturated gas solutions is contemplated by this disclosure. The device being used for any non-medical need where a highly saturated gas in solution is needed is contemplated by this disclosure.

These and other important objects, advantages, and features of the invention will become clear as this disclosure proceeds. The invention accordingly comprises the features of construction, combination of elements, and arrangement of parts that will be exemplified in the disclosure set forth hereinafter and the scope of the invention will be indicated in the claims.

### SHORT DESCRIPTION OF FIGURES

FIG. 1 discloses an illustrative embodiment of the claimed depicting gas, liquid, and solution flows.

FIG. 2 discloses a preferred geometry within a rotary mechanical phase contactor.

FIG. 3 discloses prior art geometry within a rotary mechanical phase contactor as described in U.S. Patent Application 20160015739.

FIG. 4 shows a graph proving that bubble size and gas/liquid interfacial area are inversely related.

FIG. 5 shows a graph depicting the Energetics of Surface Area Production.

FIG. 6 shows a graph depicting the resulting equilibrium relationship between oxygen pressure and dissolved oxygen concentration in water at 95° F.

FIG. 7 shows a graph of Dissolved Oxygen/Oxygen Pressure Equilibrium with supersaturation isobars.

$${( \frac{Δ P}{L} )}_{T R}$$

FIG. 8 is a summary table showing results for turbulent rectilinear and turbulent toroidal

$${( \frac{Δ P}{L} )}_{T T}$$

flows.

FIG. 9 is a graph showing DO f (MFC Oxygen Flowrate at 6.8 LPM).

### DETAILED DESCRIPTION

In the following detailed description, reference is made to the accompanying drawings, which form a part thereof, and within which are shown by way of illustration specific embodiments by which the invention may be practiced. It is to be understood that other embodiments may be utilized and structural changes may be made without departing from the scope of the invention.

As used in this specification and the appended claims, the singular forms “a”, “an”, and “the” include plural referents unless the content clearly dictates otherwise. As used in this specification and the appended claims, the term “or” is generally employed in its sense including “and/or” unless the context clearly dictates otherwise.

As used herein, “about” means approximately or nearly and in the context of a numerical value or range set forth means±15% of the numerical. In an embodiment, the term “about” can include traditional rounding according to significant figures of the numerical value. In addition, the phrase “about ‘x’ to ‘y’” includes “about ‘x’ to about ‘y’”.

Further, any range of numbers recited in the specification or claims, such as that representing a particular set of properties, units of measure, conditions, physical states or percentages, is intended to literally incorporate expressly herein by reference or otherwise, any number falling within such range, including any subset of numbers within any range so recited. For example, whenever a numerical range with a lower limit, RL, and an upper limit RU, is disclosed, any number R falling within the range is specifically disclosed. In particular, the following numbers R within the range are specifically disclosed: R=RL+k*(RU−RL), where k is a variable ranging from 1% to 100% with a 1% increment, e.g., k is 1%, 2%, 3%, 4%, 5% . . . 50%, 51%, 52% . . . 95%, 96%, 97%, 98%, 99%, or 100%. Moreover, any numerical range represented by any two values of R, as calculated above, is also specifically disclosed.

In certain embodiments of the invention, disclosed is a device and method that can produce an oxygenated solution which ultimately can be delivered to a patient, whether animal or human, without the need to expose the patient to a pressurized oxygen environment. As discussed in the background, the avoidance of safety risks, avoidance of high capital costs, and potentially exposing a wound to an oxygen environment above 2.8 atmospheres is significant as this is the maximum conventional HBOT operating pressure. While oxygen and water have been shown to have healing properties, the device can function to make any gas saturated metastable solution for whatever is deemed appropriate. Solute gases for use in the device include but are not limited to oxygen, nitrogen, nitrous oxide, nitric oxide, carbon dioxide, hydrogen. Mixtures of these gases are also contemplated by this disclosure including air. Solvents for use in the device include but are not limited to water, phosphate buffered saline (PBS), Wisconsin Solution, and proprietary organ preservation solutions such as Viaspan®, STEEN Solution™, and Perfadex®. The latter proprietary solvents have potential applications in limb and organ preservation.

While certain embodiment(s) focus on creating an oxygenated solution using O₂ gas and water, the device and method as described are not limited to this solute and solvent; the description below is merely illustrating O₂ gas and water as examples and for showing data throughout. The device and method described and claimed may be used with essentially any chemically compatible combination of gas and liquid that is capable of forming a solution. For embodiments that use water as the liquid typical water sources such as tap water, distilled water, deionized water R—O water, etc. can be used emanating from their typical sources using their typical flow hook-ups. Suitable sources of oxygen include but are not limited to pressurized gas cylinders, cryogenic sources, and portable oxygen concentrators as becoming ubiquitous in-home medical care.

Dissolved oxygen (DO) concentration units of mg O₂ (STP)/1-water will be used as a convention. Accurate and precise measurement of DO concentration can be challenging at elevated concentrations. Analytical methods using three different types of probes are used to measure DO concentration: polargraphic probes, galvanic probes, and optical/fluorescence probes. In certain embodiments, optical/fluorescence probes measured the elevated DO concentrations. One commercially available probe is capable of measuring an upper limit of 90 mg/l. Since this invention is capable of DO concentrations well in excess of 90 mg/l, the optical/fluorescence probe are used with a technique modification. Fluorescence does not consume oxygen during the measurement as is otherwise the case with the other two probes. An oxygen depleted boundary layer on the probe membrane surface is therefore avoided when an optical/fluorescence probe is used. The oxygen depleted boundary layer gives rise to artifactual results. A dilution method is used in situations where DO values are attained that exceeded the measurement capability of even the highest value-range commercial fluorescence probes. A simple weighted average calculation considering the DO value of the diluent provided reproducible results where DO₂=fₓDOₓ+f_{y}DO_{y} where x=as produced oxygen solution, y=diluent solution, and z=solution of unknown DO. Using this method, the DO concentration of metastable solutions produced by certain embodiments of the device are measured in as being excess of 250 mg/l, as a non-limiting example.

Benefits of healing have been seen with at least 30 mg/l concentration of oxygen. 38 mg/l represents the a fully saturated DO level at STP. DO concentration will be measured by using a fluorescence DO probe such as, but not limited to, a YSI Instruments ProODO optical DO probe. DO concentration measured in metastable solutions produced by certain embodiments is at least about 90 mg/ml, the maximum level that can be detected by commercial probes.

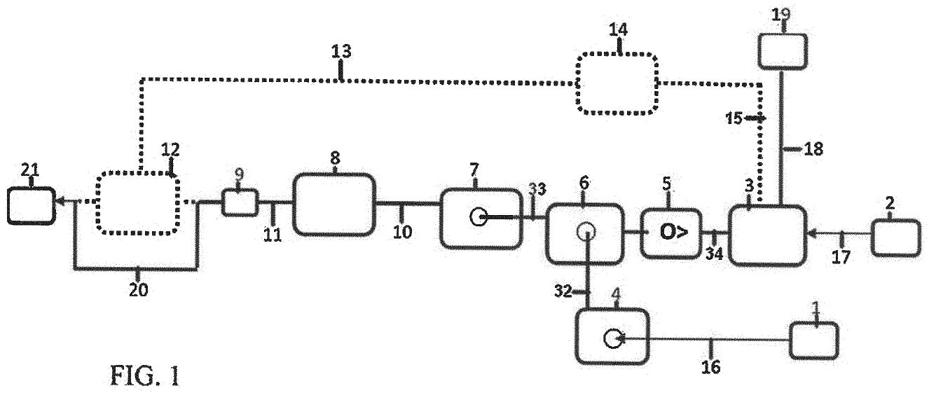

FIG. 1 discloses an illustrative embodiment depicting gas, liquid, and solution flows. Any particular physical arrangement or setup of the disclosed equipment that is functional is contemplated by this disclosure. In this non-limiting example system, water and oxygen are used as the solvent and solute gas, respectively. Water source 1 is fluidly connected to densification pump 4 via water inflow 16. Oxygen source 2 is fluidly connected to mass flow controller 3 via oxygen inflow 17. Water source 1 and oxygen source 2 are not part of the device per se, typically they are used in conjunction with the device, though it is contemplated herein to have them integrated into the device as well. Any feasible gas source and liquid source is contemplated by this disclosure. Densification pump 4 and mass flow controller 3 are fluidly connected to the phase contactor 6 via water outflow 32 and pressurized gas outflow 34. Check valve 5 is placed on the pressurized gas outflow 34 of the mass flow controller 3 as a precaution to prevent water backflow through it if a negative pressure differential develops. The symbol within the box depicting check valve 5 illustrates that left to right backflow is prevented. Phase contactor 6 is then fluidly connected to pressurization pump 7 via dispersion outflow 33. Fluid issuing from phase contactor 6 is predominantly a two phase gas/liquid dispersion as will be characterized in detail as this specification continues. Pressurization pump 7 creates the conditions necessary for solubilization of the previously dispersed solute gas in the two phase gas/liquid dispersion to achieve the desired concentration as a dissolved component. Exiting nascent solution outflow 10 from pressurization pump 7 is a stable homogeneous nascent solution at the pressure created by pressurization pump 7. Pressurization pump 7 is fluidly connected to the stepless depressurization tube 8 via this nascent solution outflow 10. Depressurization tube 8 fluidly connects to the optional in-line Dissolved Oxygen (DO) probe 12 via metastable solution outflow 11. The solution within the metastable solution outflow 11 issuing from depressurization tube 8 is ideally a homogeneous fluid with a dissolved solute gas concentration that is highly saturated as determined by pressurization pump 7 and that remains highly saturated after traveling through the depressurization coil 8 at or near atmospheric pressure; ergo, metastable. Stopping valve 9 is used to impose backpressure on pressurization pump 7 for system diagnostic purposes only and is fully open during normal operation. Optional DO probe 12 may inserted in-line to monitor the amount of oxygen that is in-solution using real-time monitoring. If present, DO probe 12 generates an output signal 13 indicative of the amount of dissolved oxygen in solution, and provides this signal to optional controller 14. Controller 14 compares the measured DO value with a setpoint and creates output signal 15 through a suitable control algorithm if a deviation is present. This output signal 15 subsequently becomes an input signal to mass flow controller 3 to regulate flow of the oxygen source to phase contactor 6. If the optional control loop comprising of DO probe 12 and controller 14 is not present, the system can be operated in open loop mode by supplying fixed input signal 18 (i.e.: voltage) from power source 19. Finally, oxygenated solution 20 issues from the system to final container 21. Final container 21 is not part of device per se, typically, it is any container that the user wishes to capture the metastable solution such as a tub or any relevant container, though it is contemplated herein to have it integrated within device itself as well.

The device utilizes a “interstage gas introduction” design. The phase contactor 6 is placed between densification pump 4 (which pressurizes liquid only) and pressurization pump 7 (which creates the nascent solution from the dispersion mixture outflow from the phase contactor 6) of the device. In the claimed device, the phase contactor 6 is the means to introduce solute gas or a combination of blended solute gases to pressurized liquid ultimately forming a dispersion. A significant benefit is afforded by placing the phase contactor 6 between densification pump 4 and pressurization pump 7 because this allows the use of a relatively low gas introduction pressure. Solute gas introduction at this location requires that the solute gas pressure only need be sufficient to overcome the densification pressure issuing from the densification pump 4. The benefit of interstage gas introduction is using the work of the pressurization pump 7 to pressurize both solute gas and solvent liquid in lieu of a separate compression device to overcome solubilization. An additional benefit to a lower solute gas pressure as provided by interstage gas introduction is higher available gas volume if a compressed gas cylinder is used. Preferably the gas enter the rotary mechanical phase contactor through a tuyere, optionally a small diameter tube incorporating an orifice at its exit could be substituted for tuyere.

Mass flow controllers (MFC) can establish and maintain a flowrate of gas (oxygen) based on a set point. One example of a such a mass flow controller is the BROOKS GF-125C. The BROOKS GF-125C mass flow controller has an integrated katharometer based mass flow meter (MFM), a proportional controller, and a proportional valve with pressure compensation to provide precise closed loop control of oxygen flow at flow rates in the 500 sccm range. If oxygen is the gas and water is the liquid, the gas introduced into the phase contactor 6 at a pressure of at least 3 psig above the pressure of incoming water source 1 for the device to effectively function, and at least 25 psig above the pressure of incoming water source 1 to optimally function.

If the gas source is able to provide a metered/controlled gas at introduction pressure, an MFC will not be needed and the rotary mechanical phase contactor directly fluidly connected directly to the gas source. However as seen below, the MFC may be used in a closed or open loop approach to controlling oxygen flow. When the device is used for creating oxygen saturated metastable solutions, the oxygen levels reached at least about 90 mg O₂/l-water, the maximum level that can be detected by commercial dissolved oxygen probes, such as a YSI Instruments ProODO optical DO probe.

One significant improvement over previous device designs includes, but is not limited to, the rotary mechanical phase contactor methodology (phase contactor 6), which will be used in all described embodiments. The functional role of the rotary mechanical phase contactor is to combine the separately incoming feed streams of gas and liquid into a single gas/liquid dispersion. In the case of all described embodiments, gas is the dispersed phase and liquid is the continuous phase. The dispersion created by this rotary mechanical phase contactor is capable of efficiently forming a metastable solution upon subsequent pressurization and stepless depressurization. Qualitatively, such a dispersion will characteristically have a uniform distribution of very small diameter gas bubbles surrounded by the liquid.

Rotary mechanical phase contactors have been used in the past for other similar purposes. Once such device is a liquid-liquid rotary phase contactor cited in US 20160030469 that is used for creating a near emulsion of silicone with a water-based solution resulting in a lotion/gel end product exiting the phase contactor. This phase contactor is designed to create high shear between two liquids of similar density (not designed for gas and liquid dispersion described below) and is of substantially different mechanical design than the rotary mechanical phase contactor of this disclosure. As such for the previously described liquid-liquid rotary phase contactor, the entry point for the secondary (added) liquid is through a passage concentric with the impeller drive shaft that rotates in the continuous phase (primary) liquid. Buoyancy forces are proportional to the difference in primary/secondary liquid densities. In the case of 1 cm3 volume spheres as a dispersed or secondary phase, the buoyancy forces are 196 dynes and 980 dynes respectively for liquid-gas and liquid-liquid systems in the context that is described in US 20160030469. A buoyancy force ratio of almost 5 requires that the secondary gas phase be introduced in a manner as to remain intimate with the rotating impeller and not separate by buoyancy forces. These forces can also be considered as body forces and therefore subjected to the centrifugal force created by the rotating impeller. In the liquid-liquid phase contactor design using a concentric secondary phase feed, the secondary gas phase would migrate to the center of rotation and coalesce into a large volume of gas, which is undesirable in the device of the claimed invention.

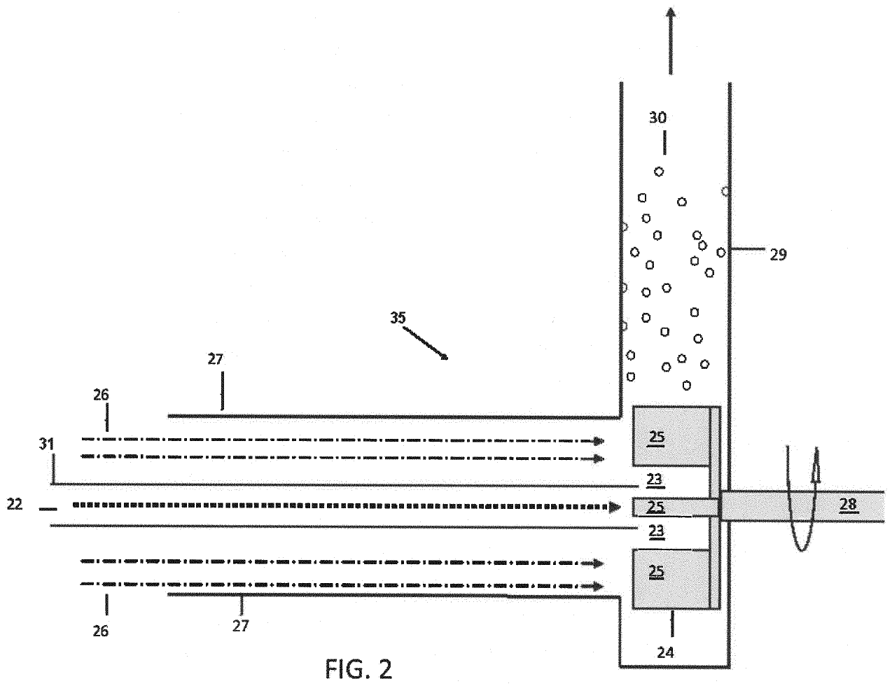

To discourage centrifugal force driven gas migration and ultimate separation, the impeller of the claimed invention was designed to enable continuous phase liquid flow to occur through the center of rotation or about the center of rotation as compared to the liquid-liquid phase contactor, as previously referenced. In this preferred geometry as unmixed solvent liquid and solute gas flow through a coaxial entry and are concurrently introduced into the center of rotation or about the center of rotation of the phase contactor impeller. The impeller may rotate at a peripheral velocity of about 80 cm/sec to in excess of about 1,100 cm/sec contingent on fluid temperature and desired solute gas concentration. An exemplary geometry within a rotary mechanical phase contactor 35 is illustrated in FIG. 2. Solute gas feed tube 31 preferably introduces solute gas flow 22 directly into the rotational center (eye) 23 of impeller 24; parallel to the rotational axis of the impeller drive. Liquid inflow tube 27 is disposed parallel to the rotational axis of the impeller drive rather than normal to this axis as in the previously referenced phase contactor. This arrangement concentrates the total volume of solute gas within the physical confines of the impeller 24 for maximum energy coupling between rotating vanes 25, solute gas flow 22, and solvent liquid 26. In this manner solute gas 22 cannot flow around the perimeter of the impeller 24 and circumvent it. The impeller is also closely coupled to motor drive shaft 28, in the preferred embodiment, with dimensions that limit fluid residence time and therefore preclude separation of the dispersed gas from the continuous phase liquid. The dispersion mixture 30 produced leaves via a dispersion mixture outlet 29 normal to the rotational axis of the impeller drive. Geometries other than this preferred embodiment are possible and perhaps even acceptable, albeit with lower performance, though contemplated by this disclosure.

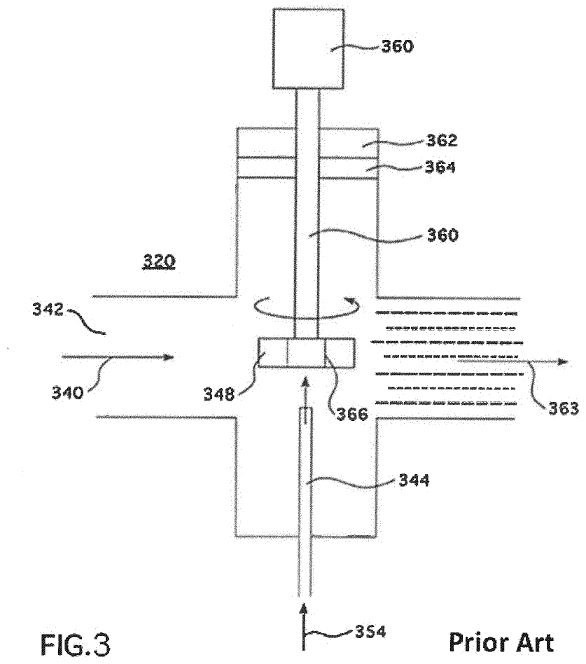

One such prior art geometry is described in U.S. Patent Application 20160015739 as shown in FIG. 3. Liquid 340 enters the body of the phase contactor 320 through conduit 342. Drive motor 360 rotates shaft 350 supported by bearings 362 and 364 with an attached impeller 348 with local radial projections 366. As impeller 348 rotates within liquid 340, turbulent eddys are created behind the trailing edges of radial projections 366. These eddys interact with gas 354 that is introduced through tube 344. This interaction results in a reduction of the stream consisting of gas 354 into small bubbles of high surface area. The movement of liquid 340 through the body of phase contactor 320 displaces the bubbles and produces the dispersion mixture outflow 363. As disclosed in this application, both solvent liquid inflow and dispersion outflow are normal to the rotational axis of the impeller drive. This phase contactor in U.S. Patent Application 20160015739 uses a solid impeller that resembles a gear and is often referred to as an R-3 Rushton Flat Disk Impeller. Such an impeller has a solid hub. The solute gas feed tube terminates below the impeller and gas inertia augmented by a small buoyancy force contribution are responsible for solute gas introduction to the eddys created by the rotating impeller. The resulting shear creates gas/liquid surface area. This geometry was prevalent until the rotary mechanical phase contactor described in the preferred embodiment was developed.

One object of the mechanical rotary phase contactor in the claimed device is to produce a dispersion comprised of small diameter bubbles from an incoming stream of solute gas such as oxygen. Another object is to produce a high density (densified) dispersion with high specific surface area; ergo, high gas/liquid interfacial area for a given unit volume of dispersion. This characteristic of a desirable dispersion will be later described as “densified” and comprehensively discussed in a subsequent section.

The objective of creating such a densified dispersion is to allow for rapid solute gas dissolution to create the nascent solution in the pressurization pump. Rapid solute gas dissolution provides a high process throughput of gas and liquid to produce a solution within the residence time characteristics of the device. This allows for the claimed device to be preferably be oriented in a compact configuration. This also allows for the claimed device to create solutions with near theoretical solute gas concentration.

Descriptions in the previous literature regarding oxygen bubbles in water frequently refer to oxygen bubbles as “micro-bubbles” or a “micro-bubble” plume. Importantly, such bubbles in the previous literature are intended to co-exist with the liquid water phase within the previous final end product bath being prepared for some subsequent use. Since the terminal velocity (or rise velocity) of small bubbles (Reynolds Number<1) is proportional to the square of the bubble radius, it is properly recognized that microbubbles are preferred in these previous solutions because these bubbles enjoy a comparatively long residence time in the liquid phase before reaching the bath surface and exiting the system. It must be emphasized that references to bubbles, microbubbles, and dispersions in context of the invention pertain to an intermediate state within the device to ultimately produce an oxygen-water solution, Microbubbles are part of the intermediate state dispersion because of favorable dissolution kinetics characteristics and other attributes that will be subsequently explained. Bubbles are not in the end product, i.e. the metastable solution produced by the device after the stepless depressurization.

The mechanical rotary phase contactor (described more below) creates a dispersion that is kinetically best suited for subsequent dissolution of the dispersed gas when it contains the highest gas/liquid interfacial area (and mass) of dispersed gas for a given volume of the dispersion. Specific surface area is a parameter that characterizes the quality of the dispersion. Since surface area (SA) is expressed in terms of length squared (l²) and volume (V) is expressed in terms of length cubed (l³), the specific surface area (SAN) of the dispersion therefore has the units of reciprocal length, i.e.: cm⁻¹.

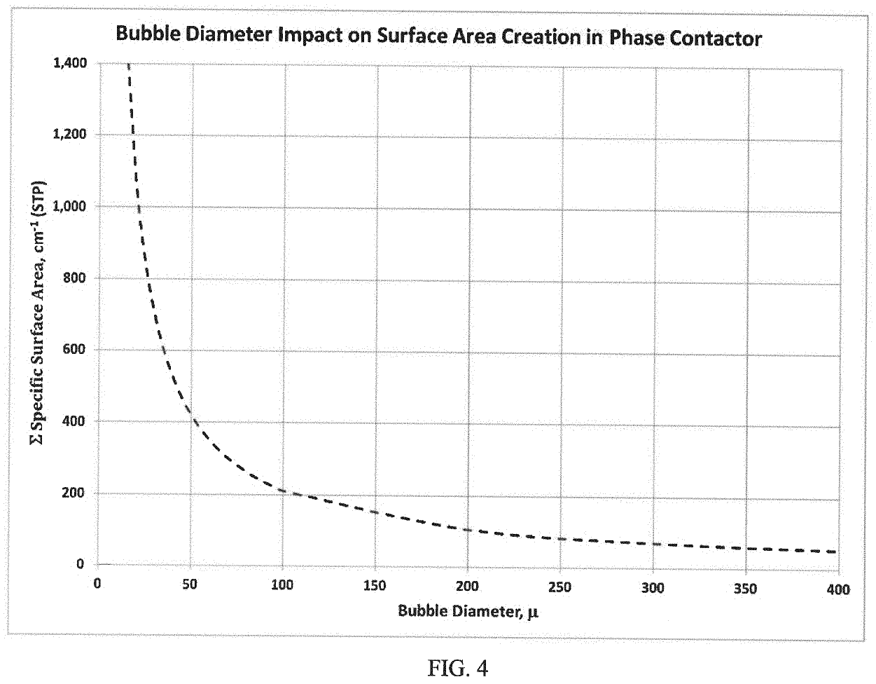

Bubble size and gas/liquid interfacial area are inversely related. The functionality of this relationship is shown as a graph in FIG. 4 where a statistical average diameter of bubbles within the dispersion is related to specific surface area; that is, the amount of gas/liquid interfacial area present in a particular volume of dispersion. Bubble diameters less than 100 microns clearly provides a mathematical advantage in terms of specific surface area. Bubbles with diameters less than 700 microns are acceptable but are kinetically disadvantaged relative to smaller bubbles.

Production of interfacial area, such as in a dispersion, is an energetic process since gas/liquid interfaces are characteristically associated with an energy quantity, i.e.: interfacial energy. In this case, the units are energy (i.e.: ergs) per unit area (i.e.: cm²). Creating more interfacial area requires that commensurate energy is supplied to the system. To clarify, micro-bubbles, preferably of less than 100 microns, are used as an intermediate state gas/liquid dispersion in the formation of the solution, but ultimately will not be in the final solution produced by the disclosed method and device. Several methods to characterize bubble size within this dispersion can be used, including acoustic techniques, turbidity, photography, and separation time. In a non-limiting illustration of the separation time method, a quantity of dispersion produced by the phase contactor is carefully harvested in a transparent cylindrical vessel, such as a graduated cylinder. The time required for an easily discernible high population micro-bubble boundary (or front) to pass from a fixed point on the cylinder wall to another is measured. Terminal velocity is calculated and related to bubble size using Stoke's law. This method is not rigorous since a bubble size distribution exists and neighboring bubbles co-interfere with individual bubble trajectories even to the point of coalescence and influencing the kinematic properties of the fluid. Further, Stoke's law is generally only applicable for spherical bubbles that follow a rigid sphere model at low Reynolds number (˜1) values. This calculation also requires an iterative process. Timing errors also can be present. Bubbles of approximately 50 micron diameter generally qualify. Since terminal velocity is related to the square of bubble diameter, a reasonably broad range of velocities is encountered. Typically, values within the range of 0.05 to 4 cm/sec are encountered.

The phase contactor in particular described embodiments is a rotary mechanical device that uses mechanical energy supplied from a prime mover such as an electric motor. Importantly, a rotary mechanical phase contactor uses externally supplied energy for this purpose. Energy does not originate with the feed gas intended to be solubilized in rotary mechanical devices as it does in pneumatic contactors described for previous devices. The motor drives a high shear rate flow impeller designed to creates eddys derived from shear to produce gas/liquid surface area. The impeller in the preferred embodiment incorporates vanes that project normal from the surface of a disk and do not extend completely to the center of the disk thus forming a cavity (See also FIG. 2). In the mechanical rotary phase contactor of the preferred embodiment, feed gas is introduced at the center of rotation or about the center of rotation and within this cavity, subsequently interacting with counter-current vortices that are shed from the trailing edges of the rotating vanes.

Phase contactors used in other previous device designs to make high saturated solutions employed a purely pneumatic mechanism based on nozzles. Pneumatic phase contactors previously described that formed high oxygen solutions include porous diffusers and sparging devices. In both cases a high porosity grade (small pores) medium is used to introduce a feed gas into the liquid phase. A categorical limitation of these pneumatic phase contactors is their inherent inability to function at the energy level needed to create high specific surface area dispersions within the feed gas flowrate range required for the desired oxygen levels. Energy for dispersion used by a pneumatic phase contactor is derived from the kinetic energy and isothermal expansion energy of feed gas issuing from the nozzle. Pressure energy upstream of the nozzle is converted into kinetic and expansion energy which is subsequently converted into surface energy. Shear to produce eddys needed to create a dispersion is only available at the gas/liquid interface of the jet, and later within the vortex street as the jet dissipates. Porous diffusers create gas bubbles that represent some multiple of the pore diameter. Due to the low energy associated with this process, bubbles issuing from the diffuser rapidly coalesce with neighboring bubbles representing the system's tendency to limit surface area and reduce surface energy.

In both nozzle and diffuser based pneumatic phase contactors of previous devices, energy increases with the amount of gas (mass flow rate) passing through the contactor. The energy source for surface area creation in pneumatic phase contactors is the solute feed gas itself; i.e.: coupled. High density dispersions with high specific surface area as required for high dissolution kinetics corresponds to an unacceptably high mass flow rate of gas in this situation. This oxygen mass flow rate required by energetic considerations is disproportional to the constitutional requirements of the oxygenated solution being created. A significant aspect of the phase contactor in the claimed device is that input energy (or power, as a rate) is decoupled from feed gas flow and can therefore be increased independently (i.e.: motor power) to satisfy the energetic requirements.

In the claimed invention, specific surface area and power coupling are characteristic parameters for the dispersion and the rotary mechanical phase contactor, respectively. High specific surface area results in a dense dispersion. Power coupling is a parameter that characterizes the conversion of mechanical energy of a rotating impeller into another form of energy such as pressure (pumping) or surface energy. Heat is also produced. The rotor in the rotary mechanical phase contactor of the claimed invention is designed for high shear and not pumping. Power coupling in this context therefore characterizes the ability of the rotary mechanical phase contactor impeller to convert rotational mechanical energy into surface energy. A portion of this energy is converted to pressure from indigenous flow restrictions that exist within the phase contactor body and functions as diffusers resulting in a measurable fluid pressure rise. This somewhat parasitic conversion can be used beneficially as will be discussed later.

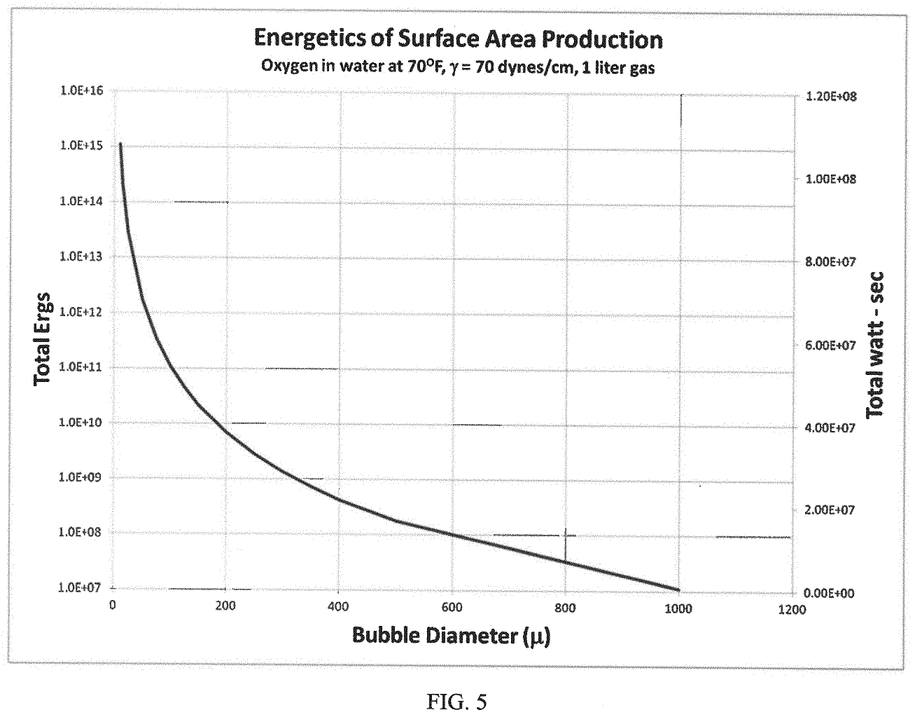

A high power coupling value is needed to create dense dispersions comprised of small bubbles and high specific surface area while avoiding large motors. The graph in FIG. 5 illustrates, as a non-limiting example, that the energetic requirement for a dispersion bubbles size reduction from 200 microns to 100 microns increases by one order of magnitude. These energy calculations are based on 1 L of oxygen. A typical feed gas oxygen flowrate for the device is 600 sccm (0.6 LPM). At 100 microns average bubble diameter, 0.6×10¹¹ ergs are needed which is equivalent to 0.12 HP-min. Since the flowrate in this example is 600 sccm, a net input power of 0.12 HP (98.5 w) is required.

As a non-limiting example, at a power coupling value of 0.8, 80% of the power input at a rotary mechanical phase contactor motor shaft is available for surface energy conversion. The remaining energy will be converted into liquid pressure and heat. Electric motors typically operate at 85% efficiency; therefore the electrical power input to the phase contactor for this example is 145 W. If the power coupling value was 0.4, however, electrical input power increases to 290 W which correspond to 0.39 electrical HP. Only 98.5 W in theory is required to create a dense dispersion. The balance of the power input will create pressure and be dissipated as heat; all unwanted parasitic losses. Clearly high power coupling values are desirable for equipment size, power source size, and heat dissipation. Aspects of this rotary mechanical phase contactor are optimized to maximize power coupling.

The terms “densification” and “densified” are important aspects of this device. Optimum dispersions have been previously described as dense. In this context, a dense dispersion is characterized by high specific surface area which, as has been shown, requires small bubbles.

Densification is a process used by the rotary mechanical phase contactor to produce a dense dispersion. The phase contactor is operated at elevated pressure and the solute gas is introduced at a predetermined pressure above the liquid operating pressure. This predetermined pressure densifies or decreases the volume of the dispersed gas required for a specified mass of the gas. The phase contactor creates a densified dispersion of liquid and gas when compared to a dispersion that would be created at STP conditions because it is configured to operate at higher than atmospheric pressure with associated higher gas density.

Densification provides three benefits: increases the volumetric efficiency of the device by producing a gas/liquid dispersion with high specific surface area (gas/liquid interfacial area for a given volume of dispersion or mass of dispersed gas, as before), increases power coupling of the rotary mechanical phase contactor by increasing the viscosity of the gas/liquid dispersion and increases the operational tolerance of the downstream pressurization pump by avoiding “cavitation”; all of which will become more apparent through the following explanations.

Volumetric efficiency of the rotary mechanical phase contactor is the ratio of the actual mass of solute gas (examples of oxygen embodiments are shown and described for illustration purposes only) processed into a dispersion relative to the total volume available for solute gas. This device operates as a constant volume process since the dominant fluid (water in this case) is essentially incompressible. The design is also of fixed geometry; therefore the hydraulic and geometric volumes are equivalent. As a non-limiting example, if the fluid residence time in the rotary mechanical phase contactor is 0.7 seconds and the liquid flowrate is 6.5 liter per minute (1 pm), the effective volume of the rotary mechanical phase contactor is 76 cm³. If the dispersion is 45% gas fraction, the volume available for gas is 34 cm³. At atmospheric pressure and an oxygen STP density of 1.43 g/L, only 0.05 g of oxygen can be accommodated. If the rotary mechanical phase contactor is designed to operate under densified conditions allowing a gas introduction pressure of 155 psig, however, the oxygen mass processed by the rotary mechanical phase contactor increases to 0.58 g. The volumetric efficiency of the rotary mechanical phase contactor is correspondingly increased through densification by a factor of 11.6.

Power coupling in the rotary mechanical phase contactor increases with densification. The bulk Newtonian viscosity of a simple liquid (such as water) containing dispersed gas can be calculated by a weighted average of the constituent liquid and gas Newtonian viscosities. Since water and gas viscosities differ by two orders of magnitude, bulk Newtonian viscosity practically becomes inversely proportional to the dispersed gas fraction. At pressures approximately less than 10 atmospheres, gas viscosity is independent of pressure. Within the Newtonian viscosity range of water, the ability of the rotating impeller to convert mechanical energy to surface area in the rotary mechanical phase contactor is a function of shear stress at the impeller vanes. At low Reynolds Numbers, this functional relationship is given by Newton's Law of Viscosity. The impeller in one embodiment of the rotary mechanical phase contactor operates at Re=170,000; well above the laminar region. Even in turbulent flow conditions, however, the relationship between impeller power and shear stress is direct, although not linear.

Since impeller power input increases with increasing shear stress, shear stress increases with viscosity, and viscosity decreases with increasing dispersion volumetric gas fraction, impeller power input decreases with increasing dispersion volumetric gas fraction. As previously shown, densification increases the volumetric efficiency of the rotary mechanical phase contactor and densification reduces the dispersion gas volume fraction for a given mass of gas introduced. Power coupling is therefore increased by densification in the rotary mechanical phase contactor.

Some embodiments of the claimed invention may have a direct fluid connection between the rotary mechanical phase contactor and the liquid source, if the liquid source can provide the liquid at a high enough pressure. However, it is anticipated that in most cases a densification pump will be needed. The densification pump of the claimed invention is used when the liquid from the liquid source does not provide the minimum acceptable liquid inlet pressure to the rotary mechanical phase contactor. The densification pump is preferably is a self-priming positive displacement device, however centrifugal and regenerative turbine pumps that are self-priming can alternatively be used. Self-priming means that the pump does not require a positive pressure at the fluid inlet to begin pumping. The output of densification pump can be optimized by using a three-phase pump motor and appropriate variable frequency drive (VFD). In this context optimization means precisely establishing a particular densification pump output pressure by varying pump speed instead of using mechanical means such as relief valves. The three-phase/VFD combination also saves energy and reduces heat because no fluid recirculation occurs, such as is the case with a pressure relief device. A preferred embodiment uses 240 V pumps and hardware because such pumps use relatively low current and are therefore smaller and of less weight as compared to their lower voltage (120 V) counterparts. A step-up transformer can be employed to convert 120 V power to 240 V as a separate unit from this device. The densification pump ensures that an appropriate positive pressure exists at the inlet of the rotary mechanical phase contactor, thus creating a higher than STP pressure environment within rotary mechanical phase contactor which further densifies the solute gas coming through the tuyere. For example, influx water to be oxygenated enters the system through water inflow and is pressurized to, for example, about 30 psig by the densification pump.

If for example, the densification pump operates at a pressure ratio of 3.0, the local liquid pressure of the water will be about 44.1 psi (absolute) or 29.4 (gauge). Correspondingly, the mass of a gas at this pressure can be easily calculated by the product of its STP density and the pressure ratio of the pump. For example, if the STP density of oxygen is 1.43 g/liter, and the densification pump operates at a pressure ratio of 3.0, 1 liter of volume at the densification pump outlet will contain 1.43×3 or 4.29 g oxygen (naturally in the water without going through rotary mechanical phase contactor). Since the device operates under constant volume conditions, the same volume now contains more oxygen by a factor of three as compared to operating the device under STP conditions. The incorporation of densification pump therefore enables a solute gas, when introduced in the rotary mechanical phase contactor, to have bubbles with higher than STP pressure by a factor of three, thereby densifying the solute gas, thus substantially increasing the volumetric efficiency of dissolution step of the nascent solution formation process.

Densification of the liquid/gas dispersion which occurs in rotary mechanical phase contactor also increases the inlet gas tolerance of the subsequent pressurization pump. Although the various embodiments of the claimed device can operate functionally with a variety of types of pumps as the pressurization pump, a preferred embodiment uses a regenerative turbine pump as the pressurization pump. Such pumps are sensitive to gas locking (often inappropriately referred to as “cavitation”) wherein the gas bubbles dispersed within the incoming liquid coalesce and envelope the rotating turbine of the pump, decoupling it from the remaining fluid. Pumping efficiency is severely compromised. Since liquid pumping occurs under constant volume, the gas fraction of this volume can be reduced by a factor of three (staying with the above illustrative example), as compared to STP conditions, for an equivalent oxygen flow. Lower gas fraction at the pump inlet therefore extends the inlet gas tolerance of the pump.

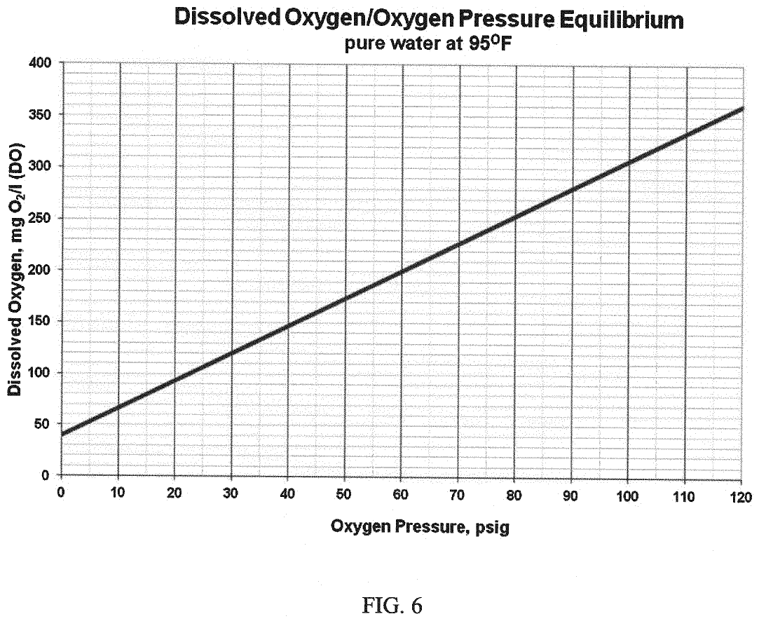

The densified gas/liquid dispersion outflow from the rotary mechanical phase contactor, the densified gas/liquid dispersion, flows via dispersion outflow to, and is pressurized by, pressurization pump to a pressure value necessary to achieve, the gas/liquid saturation conditions for the dissolved gas level of interest, which is least 1.1 STP times above the concentration at ambient saturation. When the device is used for creating oxygen saturated metastable solutions, the oxygen levels reached to at least 90 mg/l, the maximum level that can be detected by commercial dissolved oxygen probes. The following non-limiting example describes this in more detail for oxygen and water as an illustrative example only. In this step, the bubbles contained within the densified oxygen/gas dispersion are driven into solution by the elevated fluid pressure conditions created by pressurization pump. Specific pressure requirements under equilibrium conditions for particular DO (dissolved oxygen) values can be obtained by performing a Henry's Law calculation. The resulting equilibrium relationship between oxygen pressure and dissolved oxygen concentration in water at 95° F. is shown in FIG. 6, Dissolved Oxygen/Oxygen Pressure Equilibrium. Since gauge pressure is used in lieu of absolute pressure, the equilibrium line begins at a DO value of approximately 40 mg/l. This value represents fully saturated oxygen in water at 95° F. and 1 atmosphere pressure absolute.

Equilibrium considerations predict the maximum DO values attainable for a particular set of temperature and pressure conditions. The kinetics of this process also determine the final DO value achieved which is largely determined by the specific surface area of the gas/liquid interface within the densified dispersion, as previously discussed, and level of fluid turbulence created by pressurization pump. Ideally, outflow from pressurization pump is a clear liquid nascent solution at pressure representing full saturation. In this illustrative example, sight glass measurements have confirmed that full saturation has been achieved.

It has been determined that small diameter bubbles distributed within the gas/liquid dispersion issuing from phase contactor may undesirably coalesce under certain conditions. Such coalescence can negatively impact on dissolution kinetics and may impair the operation of pressurization pump in certain cases. “Close coupling” of phase contactor and pressurization pump by controlling the length of dispersion outflow minimizes this opportunity. Preferably, a separation distance of up to 24 multiples of the interconnecting tubing diameters is effective, with a tubing diameter range of about 0.125 inch to about 0.750 inch. Operational effectiveness has been maintained at separation distances as great as 25 tubing diameters and perhaps marginally more depending on tubing diameter.

Turbine and centrifugal designs are desirable for the pressurization pump because they typically operate over a variety of flow rates and maintain good efficiency. A regenerative turbine pump is preferred. Additionally, they can be “dead headed” or operate with complete outflow obstruction without damage. In some embodiments, a variable speed drive is used to adjust the outlet pressure of the pressurization pump.

The rotary mechanical phase contactor and pressurization pump function cooperatively to create fully saturated oxygen/water solution being at least 1.1×STP ambient pressure that leaves pressurization pump through nascent solution outflow to stepless depressurization tube (further described below). The rotary mechanical phase contactor can be viewed as creating the conditions most kinetically favorable for gas solubilization to occur, while pressurization pump is required to create the energetic environment necessary for the at least 1.1×STP solution to form.

Fully saturated oxygen/water solution of at least 1.1×STP exiting pressurization pump is a homogeneous, single phase clear fluid, the nascent solution. The nascent solution importantly does not contain fugitive oxygen bubbles, as these bubbles will grow at the expense of oxygen in solution. Fugitive oxygen bubbles broadly have two causal mechanisms: i) solubilization failure or, ii) undesirable nucleation of bubbles from in-solution solute gas following solubilization. Since it is an energetically preferred path to grow existing bubbles rather than to nucleate nascent bubbles from dissolved solute gas, any bubbles present in the efflux from pressurization pump are problematic and care must be taken to prevent or eliminate such bubbles.

A failure to completely solubilize the solute gas (e.g. oxygen in many of our examples) present may be caused by an energetic limitation (e.g. excess oxygen relative to equilibrium solubility at the selected temperature and pressure of operation resulting in insufficient driving force to fully solubilize the amount of oxygen present) or a kinetic limitation. In the former, equilibrium is determined by the maximum quantity of oxygen capable of being dissolved for a given set of conditions. FIG. 6 depicts the maximum solubility of oxygen in water as a function of oxygen pressure under the isothermal conditions of 95° F.

The second broad cause of solubilization failure is kinetic. A kinetic limitation may occur when sub optimal conditions do not allow sufficient mass transfer across the gas/liquid interface, due, typically, to a population of excessive bubble diameters in the efflux of the rotary mechanical phase contactor. Mass transfer occurs as a result of a flux, i.e.: mass of substance per unit time and unit surface area. Flux can be increased by more thorough mixing in the liquid or (lesser) gas phases. Such mixing increases the bulk transport component of the total diffusive flux and is optimized in the rotary mechanical phase contactor. Additional area at a constant flux also improves mass transfer. FIG. 4 effectively illustrates the inverse relationship between bubble diameter and surface area. Sufficient fluid residence time must exist in the high-pressure environment created by pressurization pump for mass transfer to occur.

Regardless of energetic or kinetic origin, fugitive bubbles present in flow from pressurization pump are highly undesirable. Since oxygen is conserved, unit mass oxygen present as bubbles will have originated from previously solubilized oxygen in the water coming from the water source. Bubbles form from solution as a result of a nucleation and growth process. Energy is required to overcome the energetic barriers of nucleation for homogeneous nucleation. Homogeneous nucleation energy may be supplied by thermal or mechanical means. Heterogeneous nucleation requires a substrate or nucleant. Such substrata include suspended particles in the solvent or certain surface-active materials. Solvent purity relative to suspended solids is an important consideration.

Pre-existing bubbles will grow more easily than if nucleated from a clear solution. A bubble growth mechanism is predominantly diffusion controlled while nucleation is both energy and diffusion controlled. Bubbles are therefore detrimental to solution integrity if present downstream of pressurization pump because they sink solubilized oxygen more easily than if oxygen nucleated homogeneously. These fugitive bubbles subsequently grow at the expense of dissolved solute gas(es)—oxygen in this non-limiting example—as previously stated.

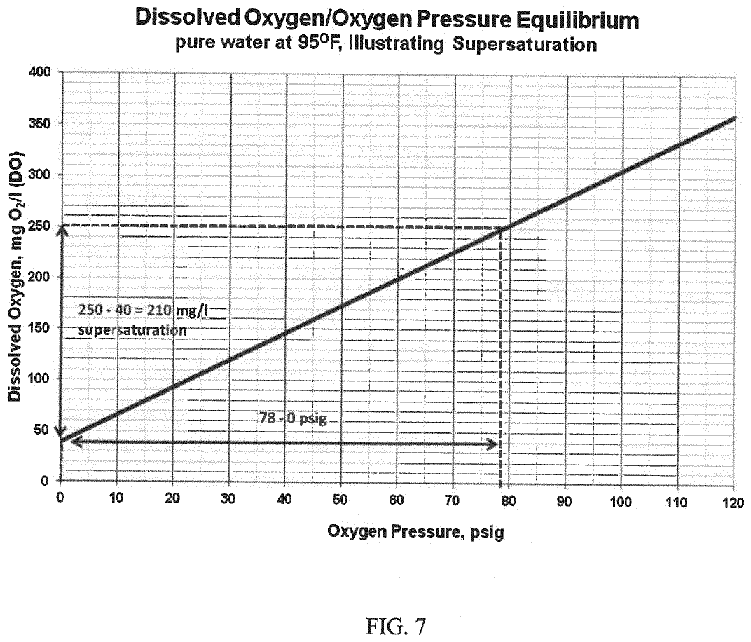

As nucleation/creation of bubbles is undesired, the proper transition from a high pressure saturated liquid present at the outlet of pressurization pump to a lower pressure where it can be usefully employed is a critical step in this process. Depressurization of a highly saturated solution to ambient conditions without losing its high gas concentration creates a supersaturated metastable solution. Such a solution is not at a state of minimum energy; it will remain in its present high solute gas concentration state provided that activation energy (or a small quantity of energy to move the system out of an energy “well”) is not supplied. Metastability is illustrated in FIG. 7 with supersaturation isobars. This graph was constructed from equilibrium solubility data and not experimentally obtained. Note that metastability is a characteristic of supersaturation; not the converse.

Still referring to FIG. 7, the graph has been annotated for use to illustrate metastability. By way of a simple non-limiting example, consider that preparation of an oxygenated solution required 78 psig of pure oxygen which resulted in a solution DO concentration of 250 mg/l. Depressurization to ambient pressure conditions for subsequent use of the solution results in a shift in equilibrium. Saturation now occurs at approximately 40 mg/l under the new conditions. This now supersaturated solution is therefore capable of rejecting 210 mg/l of oxygen if allowed to equilibrate with the new environment. Avoidance of post pressurization bubble nucleation and growth will preserve DO. It has been found that the method of depressurization can be an important aspect to the preservation of DO while in this supersaturated and metastable state.

It has been found that incorporation of traditional pressure regulators and flow control valves, such as used in previous devices, to facilitate depressurization are dubious because they create fluid turbulence that provide activation energy to the water sufficient to result in the creation of gas bubbles from a metastable supersaturated solution. Such devices have variable area orifices that generate fluid turbulence due to sudden contraction. Turbulence is proportional to the pressure drop (conversion of pressure energy to kinetic energy), degree of area change, and streamlining of the transition from a greater area to a lesser area within the intended pressure reducing device.

It has been determined that the use of a valve or series of valves to reduce the supersaturated oxygen solution pressure create turbulence that results in nucleation of oxygen gas from solution thus significantly decreasing the DO of outflow supersaturated oxygen solution. Diaphragm (sphincter) valves have demonstrated more suitably than conventional needle, globe, gate, or ball valves for preserving the DO level in the supersaturated oxygen solution due to characteristically low fluid turbulence during the passage of supersaturated oxygen solution, even when in the full open position; the latter being an important aspect.

However, in certain embodiments a stepless depressurization tube is used. The stepless depressurization tube can be employed as a stepless means to reduce the pressure of the outflow from pressurization pump to near ambient conditions while maintaining the maximum amount of dissolved oxygen in the outflow water. This is accomplished by avoiding turbulence using a controlled diameter and length tube that progressively reduces fluid pressure by viscous dissipation at the tube wall. In a configuration where this tube is coiled to create toroidal flow in a helical geometry, it will be referred to as a depressurization coil. Alternatively, rectilinear tubing can be used for stepless depressurization in the device design.

Conventional depressurization devices use one or more valves in serial flow or a back pressure regulator. All of these conventional means of pressure reduction employ a variable area orifice that introduces turbulence by form drag. Although manipulating the area available for flow is effective for flow and pressure regulation, turbulence is introduced that compromises the integrity of the dissolved gas solution. Progressive stepless depressurization, as contemplated and described herein, eliminates this problem. Depressurization of the supersaturated oxygen solution from the pressurization pump to ambient pressure will therefore occur through a stepless depressurization tube or sometimes more specifically a depressurization coil.

Actual depressurization of the supersaturated oxygen solution from the pressurization pump to ambient pressure occurs through a stepless depressurization tube. The stepless depressurization tube can be viewed upon as an infinite number of pressure-reducing valves and depends on viscous dissipation throughout its length for depressurization. The internal diameter and length of stepless depressurization tube can be determined such that it results in a near complete pressure dissipation from system operating pressure to ambient. Importantly, the conversion of fluid pressure to kinetic energy by viscous dissipation in the stepless depressurization tube is continuous and transitionless, this minimizing or completely eliminating the creation of turbulence. The supersaturated oxygen solution stream issuing from stepless depressurization tube is a clear liquid and substantially free of bubbles, i.e. the metastable solution that ultimately goes into the container.

The stepless depressurization tube is sized based on the desired pressure drop using as large of an internal tube diameter as practical while maintaining the length of the tube within reasonable values. Large diameters are desirable to maintain as low a Reynolds Number (Re) as possible. Although a casual inspection of the relationship for this dimensionless group suggests that Re is directly proportional to tube diameter, under constant flow conditions the inverse is true. After performing the substitutions for volume flowrate, velocity, and tube area, the relationship for Re expressed in terms of volume flowrate Q, diameter D, fluid Newtonian viscosity η, and fluid density p, becomes:

$$R e = \frac{4 Q ρ}{π D η} , or Re ∝ \frac{1}{D}$$

In toroidal flow situations, such as in a depressurization coil, Re will change as a function of radial position within the coil. Such an effect is ignored in this simplification. This illustration depicts the net tube only and not the associated mounting and support hardware. Once the pressure drop (ΔP) has been selected based on the design flowrate, Q, through the device, a diameter, D, for Re<1900 is calculated. This value is adjusted (downward) to use the largest practical tube inside diameter for a practical tube length. A range of large tube diameters provides the lowest value for Re, however the low associated unit ΔP necessitates long tube lengths which becomes impractical. A smaller tube diameter and correspondingly higher Re may be required as a compromise. An iterative process may be required. Once the tube length is selected, the inside diameter of the coil and number of “wraps” are determined, provided that the tubing selected can be bent to accommodate the bend radius corresponding to the assigned inside diameter. This geometry is efficient and a design expedience. A second or even third layer of wraps can be used within the coil to improve design compactness and again, offers hydrodynamic benefits. The reasonable functional upper design limit for Re is about 45,000, representing a practical compromise between unit pressure drop (Re a to ΔP) and tube length. Desirably, much lower values for Re can be used if space permits, and the converse if space is very limited. Since such values categorically provide less turbulence. Re values less than 2,100 are the most desirable; however, the length of the depressurization tube and consequential size of any means to accommodate it becomes prohibitively large. Large internal tube diameters also increase the difficulty in achieving acceptable tube bends, since tube inside diameter and bend radius are directly related. A tube diameter corresponding to Re values of 150,000 have been successfully used, albeit with reduced solution preservation performance due to fluid turbulence.

The principal aspect of stepless depressurization is utilizing a tube of appropriate inside diameter and length with an appropriate surface profile for a particular set of gasified fluid outflow conditions, that is, flowrate and pressure. Helical coil geometry offers several volumetric advantages of a pragmatic nature (including compactness), but also provides a substantial hydrodynamic benefit as compared to a linear depressurization tube operating in pure Poiseuille flow. Helical coils are volumetrically efficient in accommodating a long tube length within space limitations.

An analytical description of a straight depressurization tube immediately follows and afterwards, an analytical description depressurization coil follows.

Ignoring entrance effects, the radial velocity distribution for a fluid flowing in a fully filled straight round tube (such as a depressurization tube) with a symmetric cross section and negligible surface roughness in laminar Poiseuille flow is parabolic. As the bulk fluid velocity increases and with increasing Re, this profile flattens. In fully developed turbulent flow the velocity profile is flat.

From the energy equation, the unit pressure drop/flow relationship for a flat tube with no change in elevation is given by a form of the Fanning Equation:

$$\frac{Δ P}{L} = \frac{2 f ρ {V}^{2}}{D q} ,$$

Where

$$\frac{Δ P}{L}$$

is the pressure drop per unit length, f is the friction factor, V is fluid velocity within the tube, and D is the tube diameter. Volume flowrate Q, in terms of fluid velocity, V, and tube cross sectional area A, is:

Q=VA

In terms of tube diameter D,

$$A = \frac{π {D}^{2}}{4}$$

### Therefore,

$$V = \frac{4 Q}{π {D}^{2}}$$

Substituting,

$$\frac{Δ P}{L} = \frac{32 f ρ {Q}^{2}}{{π}^{2} {D}^{5} g}$$

For laminar flow (Re<˜2100)

$$f = \frac{16}{R e}$$

Or in terms of Q,

$$f = \frac{4 π D η}{Q ρ}$$

Where ρ is fluid density and η is fluid viscosity (note kinematic viscosity,

$$v = \frac{η}{ρ} )$$

Again substituting and simplifying,

$$\frac{Δ P}{L} = \frac{128 η Q}{π {D}^{4} g}$$

Under laminar flow conditions and for a straight decompression tube, the latter equation provides a relationship between unit pressure drop and fluid/design parameters.

An analogous relationship for turbulent flow and a strait decompression tube follows:

For turbulent flow (2100<Re<100,000)

The methodology used for the turbulent case development is similar in approach to the antecedent laminar flow situation; obtain values for friction factor and relate to pressure drop through the Fanning equation. Note that no representation for turbulent viscosity or other refinements are being used. The accuracy of these and subsequent calculations is sufficient for illustrative and even design purposes.

A correlation similar to the Blasius formula provides a friction factor for turbulent flow and Re<100,000 in a hydraulically smooth pipe. Moody Charts can also be used. This friction factor is given by:

f=0.0791Re^{-0.25}

The unit pressure drop,

$$\frac{Δ P}{L}$$

relationship becomes:

$$\frac{Δ P}{L} = \frac{2.53 ρ {Q}^{2} R {e}^{- 0.25}}{{π}^{2} {D}^{5} g}$$

Or in terms of Q (not reduced for illustration),

$$\frac{Δ P}{L} = \frac{2.53 ρ {{Q}^{2} ( \frac{4 Q ρ}{π D η} )}^{- 0.25}}{{π}^{2} {D}^{5} g}$$

Under turbulent flow conditions up to Re=100,000 and for a straight decompression tube, the latter equation provides a relationship to calculate unit pressure drop

$$( \frac{Δ P}{L} ) .$$

The case for toroidal flow through helix/coil geometry (a depressurization coil) will now be considered. Flow stability will be addressed first, followed by pressure drop prediction for toroidal flow cases. Only the relationships will be introduced, here. Once accomplished, numerical examples for flow in rectilinear and toroidal cases will be given and compared to illustrate the effects of toroidal flow.

The first effect of toroidal flow in a depressurization coil is to desirably increase the laminar—turbulent transition; i.e.: increasing the critical Reynolds Number. As shown by Ito (Ito, H. Trans ASME Vol 82D, pp 123-134, 1959), the value of the toroidal flow critical Reynolds Number, Re_{c} is given by:

$$R {e}_{c} = 20 , 000 {( \frac{D}{{D}_{H}} )}^{0.32}$$

Where D_{H}=diameter of the helix or coil to the constituent tube centerline, and D=inside diameter of the depressurization tube, as used previously.
This relationship is valid over a

$$\frac{D}{{D}_{H}}$$

range of 15-860, and will be subsequently used in an illustrative example. The effect will be to extend toroidal flow stability to higher equivalent values than a conventionally defined Re.

The second effect of using coil geometry for a depressurization tube is an increase in effective friction factor and subsequent increase in unit equivalent length pressure drop for the coil-helix configuration as compared to a rectilinear tube. Twin symmetrical countercurrent vortices induced by toroidal flow in the tube cross-section, and as formerly described, modify tube friction factor.

The Dean number, De, is a dimensionless group that represents the square root of one half the product of inertial and centripetal forces divided by viscous forces:

$$D e ≡ {[ \frac{( Inertial ) ( Centripetal )}{2 Viscous} ]}^{0.5}$$

De essentially describes the ratio of the friction factor for a curved tube (such as used in a helical geometry depressurization coil) relative to an equivalent length and otherwise identical rectilinear tube. In terms of Re and curvature ratio

$$( \frac{D}{{D}_{H}} ) ,$$

$$D e = R {e ( \frac{D}{{D}_{H}} )}^{0.5}$$

This relationship is valid in situations where a helical depressurization coil is operating in laminar toroidal flow.

The subsequent relationships for turbulent toroidal flow in a helix (depressurization coil) are valid for a De range of:

10^{1.6}<De<10³

The relationship between Re, tube diameters, and friction factors in helix and rectilinear configurations is given by Ito (ibid), and valid for

$$R {e ( \frac{D}{{D}_{H}} )}^{2}$$

values less than about 6.

$$\frac{{f}_{H}}{f} = {[ R {e ( \frac{D}{{D}_{H}} )}^{2} ]}^{\frac{1}{20}}$$

White (White, C. M. Trans. Institute of Chemical Engineering, Vol 10:66, 1932) provides an alternative relationship expressed in terms of system parameters:

$$\frac{{f}_{H}}{f} = 1 + 0.0 \; 75 {( \frac{4 Q ρ}{π D η} )}^{0.25} {( \frac{D}{{D}_{H}} )}^{0.5}$$

Using Ito, and again expressed in terms of system parameters,

$$\frac{{f}_{H}}{f} = {[ \frac{4 Q D ρ}{π η} {( \frac{1}{{D}_{H}} )}^{2} ]}^{\frac{1}{20}}$$

Recalling the Fanning equation expressed in terms of system parameters,

$$\frac{Δ P}{L} = \frac{32 f ρ {Q}^{2}}{{π}^{2} {D}^{5} g}$$

### Combining:

$$\frac{Δ P}{L} = {\frac{32 ρ {Q}^{2}}{{π}^{2} {D}^{5} g} [ \frac{4 Q D ρ}{π η} ( \frac{1}{{D}_{H}} ) 2 ]}^{\frac{1}{20}}$$

Under turbulent flow conditions up to Re=100,000 and for a straight decompression tube, the latter equation provides a relationship to calculate unit pressure drop

$$( \frac{Δ P}{L} ) .$$

This expression remains in an unsimplified form for illustration and is now explicit in system parameters (Q, D, D_{H}) and material parameters (ρ, η). It provides a value for unit pressure drop

$$( \frac{Δ P}{L} )$$

for toroidal flow in a helix as a dependent variable. The White equation previously cited could be substituted into the Fanning equation and alternatively used.

Summarizing, the following equations for unit pressure drop

$$( \frac{Δ P}{L} )$$

for rectilinear tubes in laminar and turbulent flow, and turbulent toroidal flow in a helical tube are:

### Laminar/rectilinear:

$${( \frac{Δ P}{L} )}_{L R} = \frac{128 η Q}{π {D}^{4} g}$$

### Turbulent/rectilinear:

$${( \frac{Δ P}{L} )}_{T R} = \frac{2.53 ρ {{Q}^{2} ( \frac{4 Q ρ}{π D η} )}^{- 0.25}}{{π}^{2} {D}^{5} g}$$

### Turbulent/toroidal:

$${( \frac{Δ P}{L} )}_{T T} = {\frac{32 ρ {Q}^{2}}{{π}^{2} {D}^{5} g} [ \frac{4 Q D ρ}{π η} ( \frac{1}{{D}_{H}} ) 2 ]}^{\frac{1}{20}}$$

Only the results for turbulent rectilinear

$${( \frac{Δ P}{L} )}_{T R}$$

and turbulent toroidal

$${( \frac{Δ P}{L} )}_{T T}$$

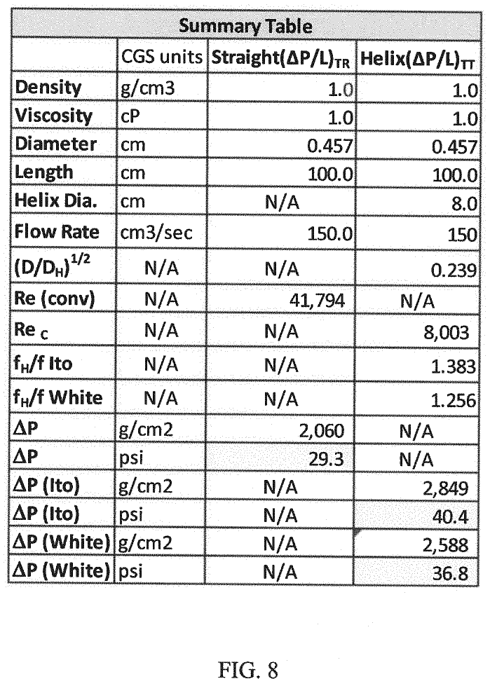

are presented in FIG. 8, since the laminar flow

$${( \frac{Δ P}{L} )}_{L R}$$

case does not apply in this practical depressurization example. The laminar case was used for illustrative purposes only in the analytical development.

Total pressure drops (ΔP) for the rectilinear (straight) and helical geometries in turbulent flow are given for water at STP flowing through a 4.57 mm (0.0180 inch) inside diameter smooth tube of 1000 mm (39.4 inches) length at a volume flow rate of 150 cm³/sec (2.4 gallon/minute). In the helical case, a coil tube center line diameter of 80 mm (3.15 inches) is considered. Re values for both cases are given as well. Re for the helix is calculated using the Re_{c} correlation as previously presented.

In the helical situation, values for unit pressure drop

$$( \frac{Δ P}{L} )$$

based on both Ito's work and White's equations are calculated. These values become total pressure drop for the depressurization tube when multiplied by tube length, as tabulated in the Summary Table.

First, it is noted that Re for the rectilinear case is almost 42,000 which clearly indicates turbulent flow. Although adequate depressurization can be accomplished at high Re in rectilinear flow while preserving the metastable gasified solution, laminar conditions are preferred. As pointed out in paragraph 063,

$$Re ∝ \frac{1}{D}$$

under constant now conditions with fixed fluid properties, however, the only means available to lower Re in the rectilinear case is to increase the depressurization tube diameter. Typically,

$$\frac{Δ P}{L} ∝ \frac{1}{{D}^{4}} ,$$

thus requiring an exceptionally long large diameter depressurization tube to provide adequate unit pressure drop while simultaneously operating at low Re. Spatial considerations will likely prohibit such a tube length unless the tube is combined with a gasified solution delivery at a suitably remote location.

Second, the total pressure drop for the rectilinear tube operated under the conditions specified in the Summary Table is calculated to be 29.3 psi. Since the tube is straight, the only mechanisms responsible for this calculated pressure drop are viscous dissipation and any turbulence within the tube.

The rectilinear tube in this example is therefore operating in turbulent at a Re of nearly 42,000 and imparting a total pressure drop of 29.3 psi.

In the helical case, the same tube under the same flow conditions as in the rectilinear case was coiled into an 8 cm diameter form. The relationship for Re_{c}, as previously given yields a value of essentially 8,000 (8,003) for this situation. Criteria for laminar—turbulent flow transitional involve both the square root of the tube to helix diameter ratio and Re, as described by H. Ito in the previous citation. The combination of

$${( \frac{D}{{D}_{H}} )}^{0.5} = 0.239$$

and Re=8,003 fall into a laminar (albeit incipient transitional) flow domain. Using the expression derived for

$${( \frac{Δ P}{L} )}_{TT} ,$$

and multiplying by the equivalent rectilinear tube length provides a total helical depressurization coil of 36.8 psi and 40.4 psi with the White and Ito correlations for turbulent friction factor, respectively. This compares to 29.3 psi for a rectilinear tube that is clearly operating in the turbulent flow regime; a 26-38% beneficial increase in pressure drop for the same tube length. Moreover, the rectilinear tube is clearly operating in fully developed turbulent for where the helical depressurization tube is transitional at worst and laminar at best.

Summarizing, a depressurization tube is an effective means to accomplish stepless progressive depressurization of a stable gasified solution to ambient conditions thereby creating a metastable gasified solution. Others are possible. The objective is to dissipate pressure energy as heat energy without turbulence that results in preservation of the metastable gasified solution. A stepless depressurization tube is an effective means to accomplish this and a helical geometry provides a significant spatial benefit in compactness. This geometry hydrodynamically creates secondary flow fields within the tube that result in higher energy dissipation and greater flow stability.

Alternate methodologies are contemplated that impart forces on fluid passing through a tube that develop into secondary flow wherein this flow results in a greater hydrodynamic pressure drop. A rectilinear tube experiencing axial rotation is one example of such methodologies. In this situation, Coriolis forces are set up that induce secondary flow similar to a helical tube and result in a higher flow resistance and ultimately greater pressure drop than an equivalent length non-rotating tube.

Though great lengths are taken to avoid bubbles and nucleation there is an awareness that any local protuberance, projection, or sudden enlargement/constriction in the outflow from the stepless depressurization tube is capable of, and likely will, provide sufficient activation energy to nucleate bubbles within the outflow stream in this depressurized and highly metastable state. Such bubbles do not necessarily indicate an upset condition with the device as described. Rather, they identify some energetic state downstream of the device (specifically the stepless depressurization tube) created by a sub optimal flow condition, and capable of nucleating bubbles. Since any nucleation and subsequent growth of bubbles occur at the expense of dissolved solute gas concentration, the intended benefits of a solution or solutions so created could be compromised. Typically, the energetic barrier to nucleation is significant. Once nucleated, however, bubble growth is a relatively spontaneous event and can proliferate to the extent that dissolved gas concentration is substantially reduced relative to the fully metastable state of a nascent solution. Such bubble growth can even “degas” a solution as a thermodynamic driving force exists to diffuse solute gas into the bubble and bring about a change of state from a dissolved species to a precipitated one.

When oxygen is used as the gas and water is used as the liquid, oxygen flow control into the rotary mechanical phase contactor is an important aspect. Below Methods 1 and 2 of controlling oxygen flow based on the final output metastable solution oxygen concentration are described in more detail. Sub-optimal oxygen flow will produce less dissolved oxygen than may be desired. Excess oxygen will compromise the efficiency of phase contactor, as gas bubble size in the dispersion will begin to increase beyond the design point maximal flow due to energy insufficiency. Extreme oxygen excess flow will result in gas locking of the downstream pressurization pump. The introduction of excess oxygen by the mass flow controller results in an energetic limitation to fully dissolve all oxygen supplied. Excess oxygen will therefore emerge from the pressurization pump as a dispersion of oxygen bubbles in a matrix of otherwise fully oxygen saturated water solvent. The check valve prevents oxygen backflow into the mass flow controller if the oxygen supply pressure becomes less than the densification pressure. Oxygen is directed into the continuous phase liquid (water) in a manner that turbulence created by the efficiently coupled rotating element in the phase contactor provides energy to create a small diameter bubble dispersion.

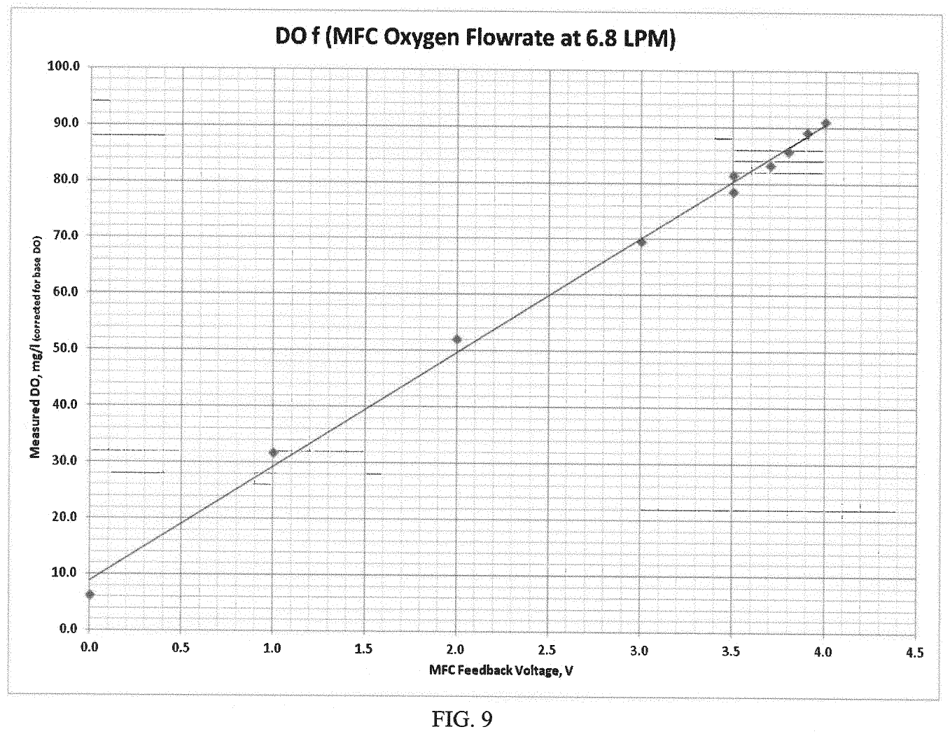

It has been shown that good linearity exists between the oxygen flowrate from the mass flow controller and the resulting dissolved oxygen (DO) value at constant water throughput. The resulting DO value is measured using the metastable solution coming from stepless depressurization tube via metastable solution outflow into final container using the DO probe. This functionality is illustrated in FIG. 9. Here, the voltage set point corresponding to a particular oxygen flowrate from mass flow controller is plotted against experimentally determined DO values measured at the outflow of the device using non-dilution methods.

Importantly, these DO values were obtained at a constant approximately 6.8 liter per minute throughput from the device. Excellent correlation is exhibited by an r² (coefficient of determination) value of 0.996, in this graphical illustration.

The oxygen flow set point of the mass flow controller can be electronically established in two ways. Method 1 uses a simple precision potentiometer (not shown) and the DO probe, which sends a controlled analog input signal that is directly applied to the mass flow controller. Any particular physical arrangement of the precision potentiometer and the device that is functional is contemplated by this disclosure. Method 1 as an open loop approach is therefore reasonable for controlling oxygen flowrate, provided other device parameters remain invariant. It does not, however, control DO values on the basis of a direct DO measurement. Predictability is lost if throughput of the device varies or other changes occur in the oxygen flow/DO relationship.

Method 2 incorporates a second proportional controller (not shown) and the mass flow controller receives its oxygen flow set point as an output from this second controller. Certain applications are contemplated wherein direct control of outflow DO is required. Method 2 adds a second control loop using a proportional controller for this purpose. Any particular physical arrangement of the proportional controller and the device that is functional is contemplated by this disclosure. This proportional controller combined with the controller/MFM/proportional valve that is integrated into mass flow controller creates the two-control loop system. The controller/MFM/proportional valve can be part of commercially available mass flow controller, such as BROOKS GF 125 from for example, though any equivalent mass flow controller that can be used in this system is contemplated for this disclosure. The controller/MFM/proportional valve incorporated within mass flow controller constitutes the first or inner control loop with the objective of maintaining an oxygen flowrate about a set point. Ordinarily the set point is used to directly set a specific oxygen flow rate as was discussed in Method 1. In Method 2, the set point is not the oxygen flow rate, but is instead, the DO value desired for a particular application. Oxygen flow rate becomes, in essence, a floating variable with the value established by the output of the second or outer control loop that controls to a DO value set point.

The proportional controller for the outer control loop receives a dissolved oxygen signal from the in-line DO probe at the outflow point of the device. This DO value is compared within proportional controller to a set point value representing the desired output DO level from the device. A deviation variable is subsequently calculated based on the difference between actual and set point DO. Based on a properly tuned standard proportional integrating derivative PID control algorithm using appropriate control parameters (i.e.: gain, integration time, etc.) used, a manipulated variable signal is provided to mass flow controller for oxygen flow rate adjustment. In essence, this outer control loop provides a set point for the inner control loop represented by the integrated mass flow controller. Method 2 is preferred because DO is measured directly and maintained in a closed loop manner using closed loop control methodologies. Other variables such as changes in the throughput of the device is automatically compensated and should not affect the outflow DO. Alternate solute gases can be controlled in this manner with the appropriate analytical probe. One such gas is carbon dioxide since accurate in-line dissolved gas probes (DGP) are well established for this gas solute. More sophisticated control methodologies such as adaptive control can be used in multi-modality treatment situations. An example of this is combinational therapy with oxygenation and phototherapy that may involve photoadaptive responses. The combination of DO and light intensity would be candidates for more complex control.

Although the descriptions above used oxygen as a solute gas and water as the solvent, the disclosed device can be used with any desired/feasible solute gas or gas mixture and any desired/feasible solvent liquid or liquid mixture. Fluid connections such as liquid inflow, gas inflow, pressurized liquid outflow, pressurized gas outflow, dispersion solution outflow, nascent solution outflow, and metastable solution outflow of the device can be any typically known in the field including but not limited to: flexible, semi-flexible, or rigid pipes, tubes, or hoses constructed of materials and strength suitable for the chemical stability and pressure tolerance. Regardless of which fluid connections are utilized, they should and will not induce turbulence. The oxygen saturated metastable solution created by certain embodiments of the device can be used in baths and to heal wounds and lesions as measured in preliminary animal studies. Alternative uses for the metastable solutions created herein also include but are not limited to oncology, ischemic conditions, limb/organ preservation among others. It further contemplated herein that the device can be used to treat any medical condition that can benefit from the use of high oxygenated or any other highly saturated gas solutions. It is further contemplated that the device can used for any non-medical need where a highly saturated gas in solution is desired or beneficial.

While specific embodiments of the claimed invention have been described in detail, it should be appreciated by those skilled in the art that various modifications and alternations and applications could be developed in light of the overall teachings of the disclosure. The exact setup/organization of the functional equipment on or off a platform is not pertinent, as the functional equipment in claimed invention can cooperate to produce a metastable solution as described regardless of the specific orientation.

It is also to be understood that the following claims are intended to cover all of the generic and specific features of the invention herein described, and all statements of the scope of the invention that, as a matter of language, might be said to fall therebetween.

1. A device comprising:
a rotary mechanical phase contactor, a pressurization pump, and a stepless decompression tube,
the rotary mechanical phase contactor being comprised of a motor and a high shear rate flow impeller incorporating vanes about a center of rotation, the rotary mechanical phase contactor being capable of fluidly connecting to a solvent liquid source and being capable of fluidly connecting to a solute gas source, both solvent liquid and solute gas being introduced into the rotary phase contactor parallel to the rotational axis of the impeller drive
the pressurization pump being fluidly connected to the rotary mechanical phase contactor via a dispersion solution outflow, the pressurization pump being fluidly connected to the stepless decompression tube via a nascent fluid outflow,
whereby the rotary mechanical phase contactor creates a dispersion of densified gas and liquid that flows normal to the rotational axis of the impeller drive into the pressurization pump, whereby the dispersion of densified gas and liquid is pressurized by the pressurization pump to create a nascent solution that is at least 1.1×STP ambient saturation, and whereby the nascent solution flows from the pressurization pump to the stepless depressurization tube, and whereby the nascent solution at that is at least 1.1×STP ambient saturation flows down the stepless depressurization tube where it is progressively depressurized to become a metastable solution that is at least 1.1×STP ambient saturation at ambient conditions.

2. The device of claim 1 wherein the rotary mechanical phase contactor creates the dispersion of densified gas and liquid by creating a gas/liquid interfacial area with bubble diameters that are less than about 700 microns.

3. The device of claim 1 wherein the rotary mechanical phase contactor creates the dispersion of densified gas and liquid by creating a gas/liquid interfacial area with bubble diameters that are less than about 100 microns.

4. The device of claim 1, wherein the high shear rate flow impeller that incorporates vanes is a disk, the vanes projecting normal from a surface of the disk such that the vanes do not extend completely to the center of the disk; thereby forming a cavity within the disk.

5. The device of claim 1 wherein the rotary mechanical phase contactor and the pressurization pump are separated by up to 24 multiples of interconnecting tubing diameters, said tubing diameters ranging from about 0.125-0.750 inches.

6. The device of claim 1 further comprising a densification pump fluidly connected to the liquid source via the liquid inflow and fluidly connected to the rotary mechanical phase contactor via a liquid outflow.

7. The device of claim 1 further comprising a mass flow controller fluidly connected to the gas source via the gas inflow and fluidly connected to the rotary mechanical phase contactor via a gas outflow.

8. The device of claim 1 wherein the pressurization pump is a regenerative turbine pump.

9. The device of claim 1 further comprising a dissolved gas probe (DGP) probe to measure the amount of gas in the metastable solution.

10. The device of claim 7, wherein an oxygen flowrate is controlled by analog input signal that is directly applied to the mass flow controller by a variable voltage or current source.

11. The device of claim 7, wherein a second proportional controller is used in combination with the mass flow controller and a DGP to create a two-control loop/closed loop control system.

12. The device of claim 1 further comprising a densification pump fluidly connected to the liquid source via the liquid inflow and fluidly connected to the rotary mechanical phase contactor via a liquid outflow; and further comprising a mass flow controller fluidly connected to the gas source via the gas inflow and fluidly connected to the rotary mechanical phase contactor via a gas outflow.

13. The device of claim 1, wherein the stepless depressurization tube is a diameter in which the Reynolds Number (Re) is less than 2,100 at a desired pressure drop and a desired design flow rate.

14. The device of claim 1, wherein the stepless depressurization tube is a diameter in which the Reynolds Number (Re) is less than 45,000 at a desired pressure drop and a desired design flow rate.

15. The device of claim 1 wherein the stepless depressurization tube is a diameter in which the Reynolds Number (Re) is less than 150,000 at a desired pressure drop and a desired design flow rate.

16. The device of claim 1 wherein the stepless depressurization tube is a helical coil.

17. The device of claim 1, wherein a feed from the gas source enters the rotary mechanical phase contactor at the center of rotation or about the center of rotation of the high shear rate flow impeller incorporating vanes.

18. The device of claim 6, wherein the densification pump is a self-priming positive displacement device.

19. The device of claim 1, wherein a solvent liquid from the solvent liquid source and a solute gas from the solute gas source flow through a coaxial entry and are concurrently introduced into the center of rotation or about the center of rotation of the high shear rate flow impeller incorporating vanes.

20. A method of producing a solution that in its nascent state is at least at least about 1.1×STP ambient saturation comprising: providing a pressurized liquid, introducing a gas solute to the pressurized liquid within a rotary mechanical phase contactor creating a densified dispersion, exposing the densified dispersion to a pressurization pump creating a solution that in its nascent state is at least about 1.1×STP ambient saturation.

21. A method of creating a solution that in its metastable state is that is at least 1.1×STP ambient saturation at ambient conditions comprising: providing a solution that in its nascent state is at least 1.1×STP ambient saturation, exposing the solution that in its nascent state is at least 1.1×STP ambient saturation to a stepless depressurization.

22. The method of claim 21, wherein the stepless depressurization is accomplished by using a helical coil.

23. The method of claim 20, wherein the rotary mechanical phase contactor uses external energy to create eddys derived from sheer to produce a gas/liquid interfacial area with bubble diameters that are less than 700 microns.

24. The method of claim 20, wherein the rotary mechanical phase contactor uses external energy to create eddys derived from sheer to produce a gas/liquid interfacial area with bubble diameters that are less than 100 microns.

25. The method of claim 20, wherein a densification pump ensures that a positive pressure exists at an inlet of the rotary mechanical phase contactor.

26. The method of claim 21, wherein the stepless depressurization comprises using a tube length for continuous and transitionless viscous dissipation.
